(19) United States
(12) Patent Application Publication
(10) Pub. No.: US 2010/0121136 A1
(43) Pub. Date: May 13, 2010

(54) Methods and apparatus for capturing and manipulating body parts

(76) Inventor: Lloyd Champagne, Paradise Valley, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 12/480,676

(22) Filed: Jun. 8, 2009

Related U.S. Application Data
(60) Provisional application No. 61/059,806, filed on Jun. 8, 2008.

(51) Int. Cl. A61B 17/00

(52) U.S. Cl. 600/37

(57) ABSTRACT

A device for grasping a body part is preferably a catheter having a proximal end and distal end, the proximal end being juxtaposed a user while in use. A grasping section is attached to the catheter near or at the distal end. The grasping section has a first position wherein a body part may be received by the grasping section and a second position wherein the grasping section grasps the body part with sufficient force to enable the operator to move the body part to a desired location.

### PRIORITY CLAIM

This application claims priority to U.S. Provisional Patent Application Ser. No. 61/059,806, filed Jun. 8, 2008, the disclosure of which is incorporated by reference in its entirety for all purposes.

### FIELD OF THE INVENTION

The present invention relates to devices and methods for retrieving and manipulating body parts, and more particularly to devices and methods for retrieving and manipulating tendons or similar body parts.

### BACKGROUND

A person's hand has tendons that extend along the length of each finger and assist in moving the fingers. When properly positioned in the finger, each tendon passes through three separate pulleys, which medical practitioners skilled in surgery understand are collar-like structures in the finger. One or more of these tendons can be accidentally severed and, if so, tension in the tendon causes the severed end of the tendon to be pulled away from the finger tip and back through one or more of the pulleys. To reattach the tendon the hand must be opened surgically, the end of the tendon retrieved, pulled back into position through each relevant pulley towards the fingertip, and sutured into place.

The difficulty in retrieving severed tendons lies largely in the problem of pulling a severed tendon back through each of the pulleys because the available space is small and current devices will not easily fit (if they fit at all) in the available space. Furthermore, many current devices are rigid and do not bend along the contour of the finger.

Therefore, a device is needed that fits through the pulleys and that can follow the contour of a finger to assist with directing tendons back through applicable pulleys. It would also be advantageous if such a device could grasp and retrieve more than one tendon at a time in order to reduce the time of a surgery.

### DEFINITIONS

“Body part” means one or more body parts, such as one or more tendons.

“Catheter” means any elongated device that may be used to extend into any part or portion of the body and used in the practice of the invention.

“Grasping portion: means any structure or device that can grasp a body part with sufficient force to move the body part into the position desired by the user.

“User” means a person using the catheter. The user may be a physician such as a surgeon.

“Wire mesh” means any structure comprised of wires or struts.

“Retriever” means to grasp a body part with enough force to move it to another position desired by a user of the device.

### SUMMARY OF THE INVENTION

Aspects of the present invention comprise a catheter with a proximal end that is juxtaposed an operator when in use and a distal end that has a grasping portion that is preferably at or near the distal end. The grasping portion is preferably comprised of a wire mesh with a first end affixed to the distal end of the catheter and a second end that has an open position wherein it does not grasp a body part and a closed position wherein it can grasp a body part with sufficient force for the user to move the body part to a desired location. The invention may be used to retrieve one or more severed tendons in the hand or used to retrieve other body parts.

### BRIEF DESCRIPTION OF THE DRAWINGS

FIG. 1 illustrates an embodiment of the present invention.

FIG. 2 shows the device of FIG. 1 with a known part attached for introducing a catheter into a body structure such as a blood vessel.

FIG. 3 shows the device of FIG. 2 with its three tubular sections separated.

FIG. 4 shows the device of FIG. 2 with the inner tube partially removed from the catheter.

FIG. 5 shows the device of FIG. 2 with the inner tube removed and the grasping portion in its open position.

FIG. 6 is close-up view of the grasping portion as shown in FIG. 5.

FIG. 7 shows the device of FIG. 5 in which the outer sheath is being moved over the grasping portion to close it.

FIG. 8 is a close-up view of the grasping portion of FIG. 7.

FIG. 9 shows the device of FIG. 5 with the outer tube/sheath covering and closing the grasping portion.

FIG. 10 is a close-up view of the grasping portion and inner tube of FIG. 4.

FIG. 11 shows the device of FIG. 1 with the stylus slightly removed.

FIG. 12 is a close-up view of a stylus and catheter tip.

FIG. 13 shows an alternate embodiment of the invention.

FIG. 14 shows a close-up view of the grasping portion of the device of FIG. 13 with the grasping portion in a partially open position.

FIG. 15 shows a close-up view of the grasping portion of the device of FIG. 13 in a fully open position.

FIG. 16 shows the device of FIG. 13 with the grasping portion in a closed position.

### DETAILED DESCRIPTION OF PREFERRED EMBODIMENTS

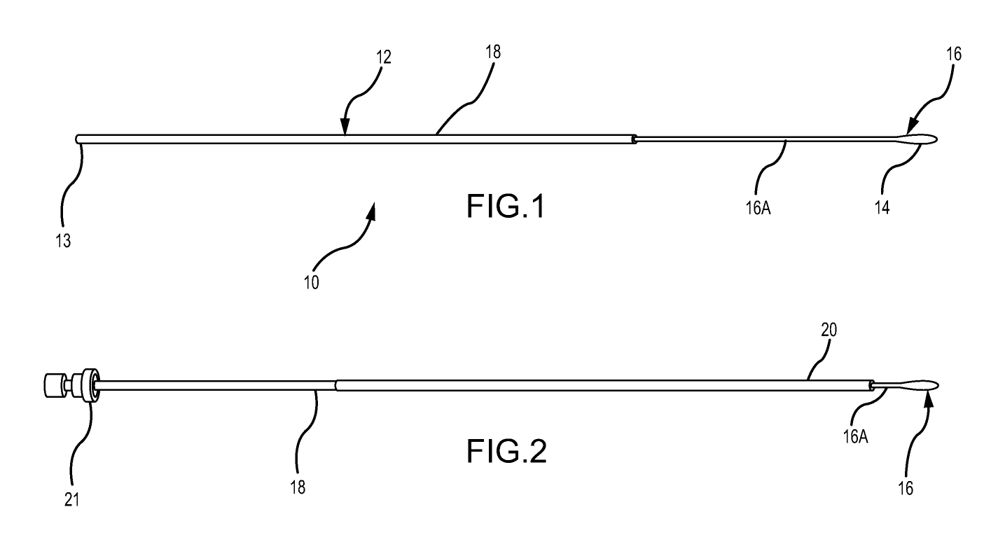

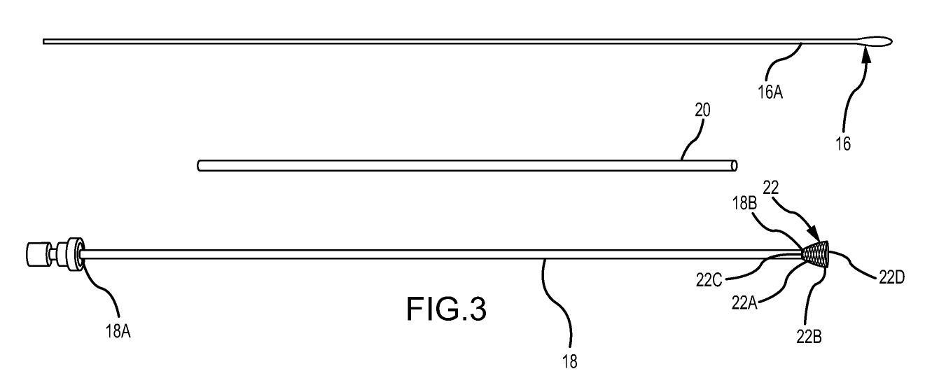

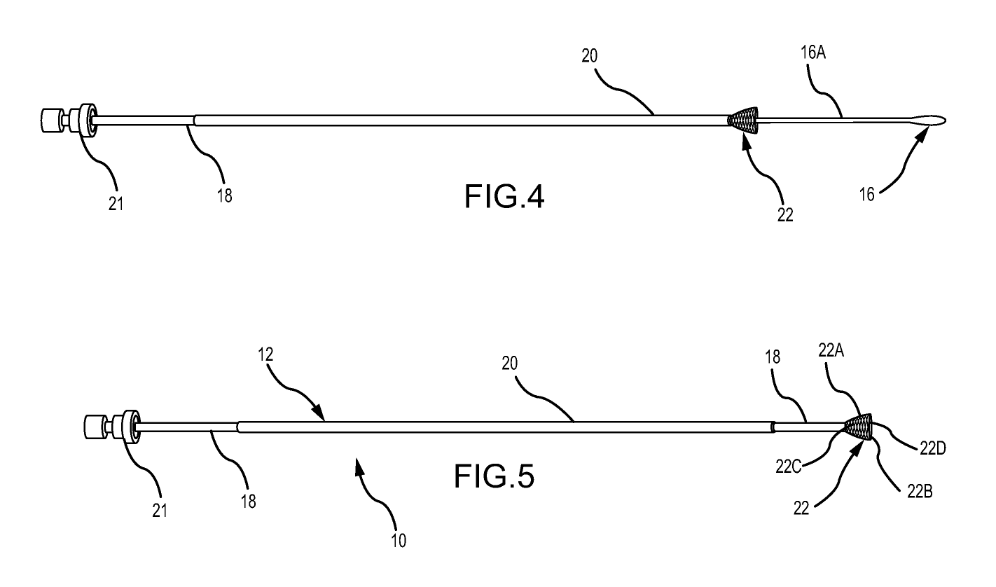

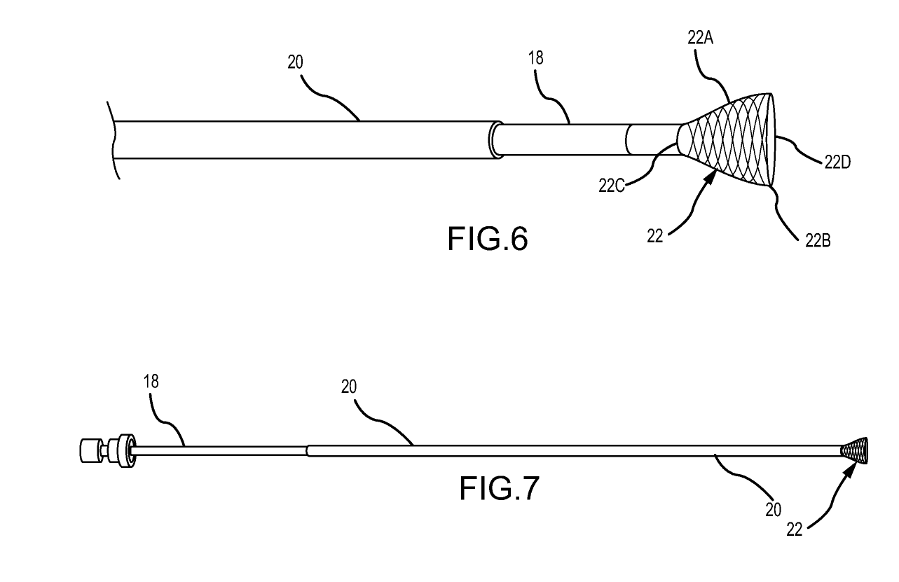

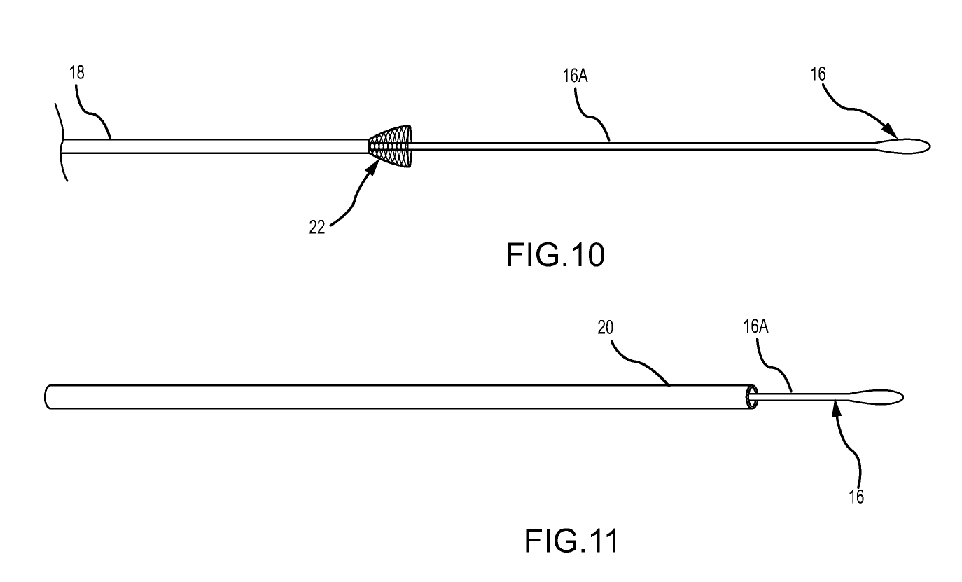

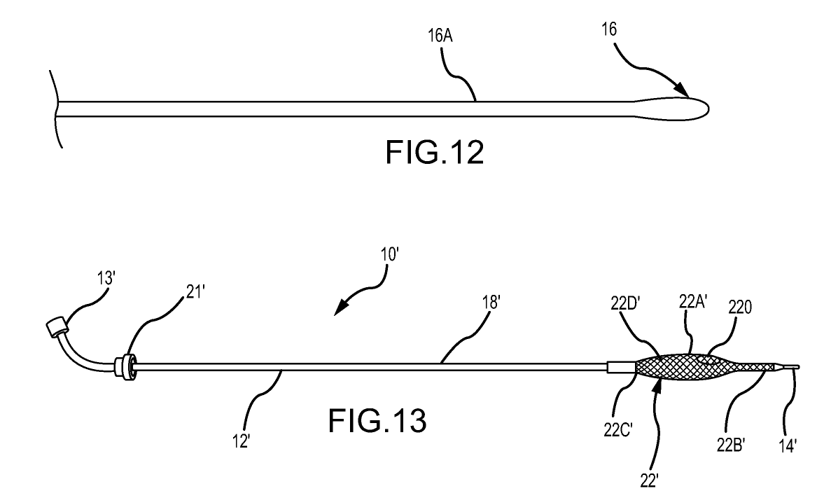

Turning now to the Figures, wherein the purpose is to describe preferred embodiments of the invention and not to limit same, FIGS. 1-12 illustrate one preferred embodiment of the invention. Device 10 comprises a catheter 12 having a proximal end 13, a distal end 14, a tip 16 (that is preferably removable), a stylus 16A attached to or formed integrally with tip 16, an inner tube 18 and an outer tube 20. A connector port 21 is preferably attached to or near proximal end 13 and is known in the art and could be any suitable type now known or designed in the future. A catheter according to the invention may have any desired diameter, but preferably has an outer diameter of between 10 mm and 25 mm.

Inner tube 18 has a proximal end 18A and a distal end 18B and a grasping section 22 is attached to (preferred) or integrally formed with end 18B. Inner tube 18 is preferably formed of biocompatible plastic and is preferably of a standard width and manufactured of standard material used to manufacture catheters.

Grasping section 22 has a proximal end 22C attached or integrally formed with end 18B, a body part 22A, a distal end 22B and a cavity 22D that is located within and defined by body part 22A when grasping section 22 is in its open position. Grasping section 22 is preferably made of wire mesh that is preferably nitinol wire mesh or stainless steel mesh, but any suitable material or device may be used for grasping section 22. Alternatively, grasping section 22 may comprise struts that are substantially parallel to one another. Grasping section 22 has an open position (as shown, for example, in FIG. 5, wherein a body part can be received in cavity 22D, and a closed position (as shown, for example, in FIG. 9) wherein grasping section 22 grasps a body part or is closed for deployment through tissue. The grasping section 22 may be provided with additional aspects that improve gripping ability, such as an inward-facing edge whether smooth or discontinuous (e.g. to have a jagged edge to improve grip), or inward-facing hooks.

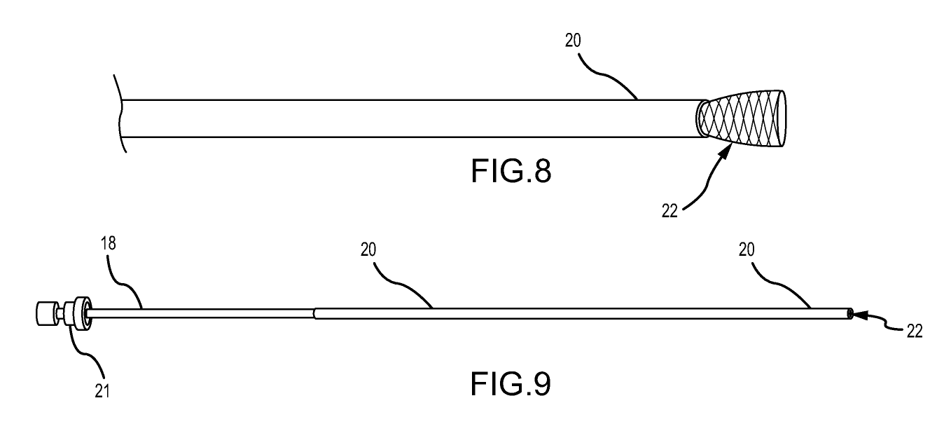

When the device 10 is assembled (see, e.g., FIG. 2), outer tube 20 surrounds inner tube 18 and may be moved over the inner tube 18 in either the proximal 13 or distal 14 directions by sliding. When the outer tube 20 is deployed in the distal direction (see, e.g. FIG. 2), the outer tube compresses the grasping section 22 and substantially reduces its effective outside diameter to a sufficient size to pass through human tissues. With the outer tube 20 in this position, the grasping section 22 is in its closed position. For example, FIGS. 7 and 8 show the outer tube 20 compressingly engaging the grasping section 22, and in FIG. 9, the outer tube 20 has completely compressed the grasping section 22 which is now nested within the outer tube Likewise, moving the outer tube 20 towards the proximal end 13 of the catheter allows the grasping section 22 to open by spring forces in the material comprising the grasping section 22 (see, e.g. FIG. 5).

As shown, the catheter may include a tip 16 and stylus 16A that is insertable into the inner tube 18 of the catheter. A purpose of tip 16 is to create a solid, rounded section that can easily be moved through certain body structures, such as pulleys in the finger or hand, and any suitable design may be used. Stylus 16A is preferably long enough to make the combined structure including stylus 16A and tip 16 easy to find and use when performing multiple procedures (for example, when retrieving the tendons on multiple fingers).

In one method of the present invention using the catheter of FIGS. 1-12, the device 10 is assembled with the tip 16 and stylus 16A is inserted into distal end 14 of the catheter, and the outer tube 20 is slid toward the distal end 14 of the catheter to compress the grasping section 22. The distal end 14 is then juxtaposed a body part to be operated upon. In one embodiment, the tip 16 of the device 10 is inserted through at least one pulley in the hand, and may be threaded through as many pulleys as needed until the tip 16 comes within the vicinity of the severed end of a tendon. Typically, the direction of insertion is from the distal end of the finger towards the patient's body, but other directions may be used depending on the body part needing to be grasped and manipulated. Once the device 10 has been inserted to a desired location, tip 16 and stylus 16A are removed. Alternatively, device 10 may not include a tip 16 or stylus 16A, or may include another type of suitable tip. Once any tip is removed, the outer tube 20 is slid toward the proximate end 13 of the device 10 to allow the grasping section 22 to open. The tendon or other tissue part is then received in the cavity 22D in the opened grasping section 22, and then the outer tube is slid toward the distal end 14 of the catheter, at least partially compressing the grasping section 22, which in turn applies a gripping force to the surface of the tendon or body part to be moved. The device 10 is then pulled from the proximate end 13 to pull the tendon or other body part, and in one embodiment, a tendon is pulled through one or more pulleys of the hand. The grasping section may then release the tissue or tendon by sliding the outer tube 20 toward the proximate end 13 of the catheter, and the physician may then take additional actions such as removing the device 10 and attaching the tendon or tissue to a desired location such as by suturing.

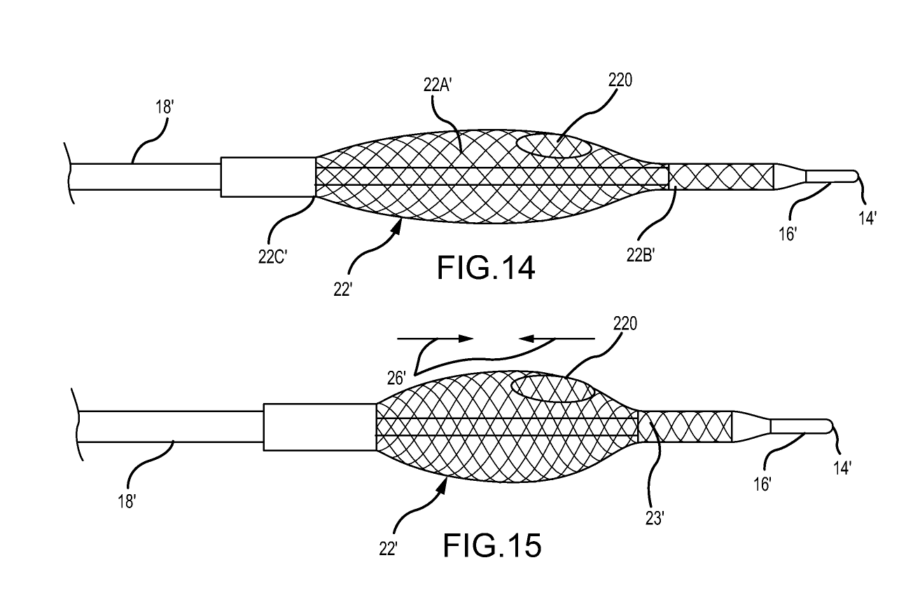

FIGS. 13-16 show an alternate embodiment of the invention. Device 10′ comprises a catheter 12′ having a proximal end 13′ and a distal end 14′. The grasping section 22′ of device 10′ is not located at distal end 14′, but is juxtaposed the distal end 14′. The catheter 12′ has an inner tube 18′ with a grasping section 22′, the grasping section 22′ having a proximate grasping end 22C′ and a distal grasping end 22B′. The proximate grasping end 22C′ is at least partially formed on or attached to the inner tube, and may in some embodiments be attached to the interior lumen of the inner tube 18′. The distal grasping end 22B′ of the grasping section 22′ is attached to an innermost tube 23′ that is slidably disposed within the inner tube 18′ and extends beyond the distal end of the inner tube 18′. The distal grasping end 22B′ may be attached to the inner most tube 23′ by any appropriate means, such as by molding, integrally forming, adhering, or coupling, and may be attached to a lumen of the innermost tube 23′.

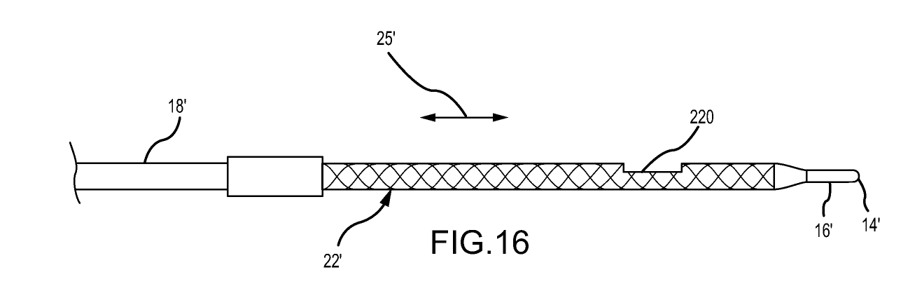

Grasping section 22′ is preferably made of wire mesh that is preferably nitinol wire mesh or stainless steel mesh, but any suitable material or device may be used for grasping section 22′. Grasping section 22′ has an open position (as shown, for example, in FIG. 15, wherein a body part can be received through an opening 220, and a closed position (as shown, for example, in FIG. 16) wherein grasping section 22 grasps and can retrieve a body part.

The innermost tube 23′ has a first position and a second position relative to the inner tube 18′, the grasping section 22′ being in its open position when the innermost tube 23′ is in its first position (see, e.g., FIG. 15, where the distal end of the innermost tube has been moved toward the proximate end of the inner tube) and being in its closed position when the innermost tube is in its second position (see, e.g., FIG. 16, where the distal end of the innermost tube has been moved away from the proximate end of the inner tube). Put another way, sliding the innermost tube 23′ through the inner tube 18′ to move the distal ends of the inner tube 18′ and innermost tube 23′ away from each other 26′ flattens and compresses the grasping section 22′ to grasp tissue or otherwise reduces diameter of the grasping section 22′ to assist with deployment through tissue Likewise, sliding the innermost tube 23′ through the inner tube 18′ to move the distal ends of the inner tube 18′ and innermost tube 23′ toward each other 25′ opens the grasping section 22′ to create an opening 220 which may receive tissue to be grasped. In other embodiments, the inner sheath 18′ may be surrounded by a slidable outer sheath (not shown) to cover the gripping section 22′ to assist with transport through tissue, or to improve the gripping strength of the gripping section 22′ when deployed toward the distal end 14′ of the catheter 12′.

A solid or flexible distal tip 16′ may also be provided that may be unremovably or removably attached to the distal end 14′ of the catheter 12′. The tip 16′ is provided to assist with threading the device 10′ through tissues to the vicinity of a tendon or other tissue that needs to be grasped and pulled.

What is claimed is:

1. A catheter for grasping a body part comprising:
a proximal end that is juxtaposed an operator when being used;
a distal end comprising a grasping section, the grasping section having an open position and a closed position thereof, the grasping section not grasping the body part when in the open position and grasping the body part when in the closed position; and
wherein when in closed position, the grasping section is configured to grasp the body port with sufficient force to enable the operator to move the body part to a desired location.

2. The catheter of claim 1 wherein the grasping section is comprised of wire mesh.

3. The catheter of claim 2 wherein the wire mesh comprises nitinol.

4. The catheter of claim 2 wherein the wire mesh comprises stainless steel.

5. The catheter of claim 2 wherein the wire mesh has a generally circular cross section between 0.006 inches and 0.020 inches in diameter.

6. The catheter of claims 2 wherein the wire mesh has a generally circular cross section between 0.008 inches and 0.015 inches in diameter.

7. The catheter of claim 2 wherein the wire mesh is formed in a criss-cross pattern.

8. The catheter claim 2 wherein the wire mesh comprises struts that are substantially parallel to one another.

9. The catheter of claim 8 wherein each of the struts is between 0.006 inches and 0.020 inches in thickness.

10. The catheter of either of claims 8 wherein each of the struts has a width of between 0.010 inches and 0.030 inches.

11. The catheter of any of claim 1 wherein the grasping section has a proximal end and a distal end.

12. The catheter of claim 11 wherein the distal end of the grasping section is open when the grasping section is in its open position and the distal end is at least partially grasping the body part when the grasping section is in its closed position.

13. The catheter of either claims 11 wherein the distal end has an inward-facing edge for grasping a body part.

14. The catheter of any of claims 1 wherein the grasping portion comprises hooks for holding the body part when the grasping section is in its closed position.

15. The catheter of claim 13 wherein the inward-facing edge is discontinuous.

16. The catheter of any of claims 1 wherein there is an opening defined in the grasping section when the grasping section is in its open position, the opening for enabling the body part to be positioned therethrough and into a cavity in the grasping section.

17. The catheter of claim 16 wherein the opening is not at the distal end of the grasping section.

18. The catheter of claim 1 that has an inner tube and an outer tube, wherein the outer tube surrounds the interior tube and is movable relative the interior tube.

19. The catheter of claim 18 wherein the outer tube has a first position wherein the grasping section is in its open position, and a second position wherein the grasping section is in its closed position.

20. The catheter of either of claim 18 wherein the outer tube does not cover the grasping section when the outer tube is in its first position, and the outer tube at least partially covers the grasping section when the outer tube is in its second position.

21. The catheter of claim 20 wherein the grasping section is in its open position when the outer tube is in its first position and the catheter is in its closed position when the outer tube is in its second position.

22. The catheter of claim 1 that has a tip at its distal end.

23. The catheter of claim 22 wherein the tip is rounded.

24. The catheter of claim 22 wherein the tip is removable.

25. The catheter of claim 22 wherein the distal tip is attached to a stylus.

26. The catheter of claim 25 wherein the stylus is removable.

27. The catheter of claim 25 wherein the stylus is positioned in a lumen in the inner tube.

28. The catheter of claim 26 wherein the inner tube has a lumen and the stylus is positioned in the lumen prior to removal.

29. A catheter for grasping a body part, the catheter comprising:
a proximal end that is juxtaposed an operator when being used;
a distal end;
a grasping section juxtaposed the distal end, the grasping section having an open position and a closed position thereof, the grasping section not grasping the body part when in the open position and grasping the body part when in the closed position; and
wherein the grasping section grasps the body port with enough force to enable the operator to move the body part to a desired location.

30. The catheter of claim 29 that has an inner tube wherein the grasping section is at least partially formed on or attached to the inner tube.

31. The catheter of claim 29 wherein the inner tube has a lumen and there is an innermost tube retained at least partially inside the lumen.

32. The catheter of claim 31 wherein the grasping section is at least partially attached to or formed integrally with the innermost tube.

33. The catheter of claim 32 wherein the innermost tube has a first position and a second position, the grasping section being in its open position when the innermost tube is in its first position and being in its closed position when the innermost tube is in its second position.

34. The catheter of claim 29 wherein the grasping section has a first end attached to or integrally formed with an inner tube and a second end attached to or integrally formed with an innermost tube.

35. The catheter of claim 33 wherein the innermost tube extends outside of the lumen of the inner tube when in its first position and extends further outside of the lumen of the inner tube when in its second position.

36. The catheter of claim 29 that further includes an outer sheath that surrounds the inner sheath.

37. The catheter of claim 36 wherein the outer sheath has a first position wherein it covers the grasping portion and second position wherein it does not cover the grasping portion.

38. The catheter of claim 29 that further comprises a distal tip.

39. The catheter of claim 38 wherein the distal tip is rounded.

40. The catheter of claim 38 wherein the distal tip is solid.

41. The catheter of claim 38 wherein the distal tip is not removable.
